(19) United States
(12) Patent Application Publication
(10) Pub. No.: US 2025/0269149 A1
(43) Pub. Date: Aug. 28, 2025

(54) MEDICAL DEVICE, AND METHOD FOR MANUFACTURING MEDICAL DEVICE

(71) Applicant: ASAHI INTECC CO., LTD., Seto-shi (JP)

(72) Inventors: Aoi OKAMURA, Seto-shi (JP); Hirokatsu TAKEMOTO, Seto-shi (JP)

(73) Assignee: ASAHI INTECC CO., LTD., Seto-shi (JP)

(21) Appl. No.: 19/209,180

(22) Filed: May 15, 2025

Related U.S. Application Data
(63) Continuation of application No. PCT/JP2022/044155, filed on Nov. 30, 2022.

(51) Int. Cl. A61M 25/09; A61L 29/02; A61L 29/10

(52) U.S. Cl. CPC A61M 25/09; A61L 29/02; A61L 29/106; A61M 2025/09133

(57) ABSTRACT

A medical device includes: a first metal member including a first metal member main body including a first element, and a coating film formed on at least a part of a surface of the first metal member main body; a second metal member; and a fixing member that fixes the first metal member and the second metal member and includes a second element that is different from the first element and is a D-block element. A method for manufacturing a medical device includes brazing a first metal member main body and a second metal member covering the first metal member main body by employing a flux and a braze material to form a fixing member that fixes the first metal member main body and the second metal member, and a coating film on a surface of the first metal member main body.

### CROSS-REFERENCE TO RELATED APPLICATIONS

This is a Continuation of Application No. PCT/JP2022/044155, filed Nov. 30, 2022. The disclosure of the prior application is hereby incorporated by reference herein in its entirety.

### TECHNICAL FIELD

The disclosed embodiments relate to a medical device, and a method for manufacturing the medical device.

### BACKGROUND

Patent Literature 1 discloses a guide wire including a metal core wire, a first coil and a second coil provided on the ends of the core wire, and a lubricating coating film that covers the core wire, the first coil, and the second coil. In the guide wire in Patent Literature 1, the both ends of the first coil and the second coil are fixed by brazing with a soft braze.

### CITATION LIST

### Patent Literature

Patent Literature 1: JP 2004-215710 A

### SUMMARY

### Technical Problem

In a medical device such as a guide wire, a core shaft main body (core wire) and a braze material are generally made of different types of metal materials. In this configuration, since the medical device is inserted into a blood vessel filled with electrolyte-containing blood during use, there is a risk that corrosion of, among a core shaft main body and a fixing member, one that is more prone to corrosion is enhanced through electronic conduction between the core shaft main body and the fixing member made of a braze material. In other words, there is a risk of galvanic corrosion. However, in Patent Literature 1, suppression of such a corrosion is not taken into consideration at all. Such a problem is common to all medical devices that are inserted into living body lumens such as lymph gland system, bile tract system, urinary system, respiratory tract system, digestive organ system, secreting gland system, and reproductive organs besides vascular system.

The disclosed embodiments have been made to solve at least a part of the above-described problem, and an object of the disclosed embodiments is to suppress corrosion of members constituting a medical device.

### Solution to Problem

The disclosed embodiments were made to solve at least a part of the above-described problem and can be embodied as the following aspects.

(1) According to an aspect of the disclosed embodiments, a medical device is provided. This medical device includes: a first metal member including a first metal member main body including a first element, and a coating film formed on at least a part of a surface of the first metal member main body; a second metal member; and a fixing member that fixes the first metal member and the second metal member and including a second element that is different from the first element and is a D-block element.

### BRIEF DESCRIPTION OF DRAWINGS

FIG. 1 is an explanatory view illustrating a configuration of a guide wire as a medical device.

FIG. 2 is an enlarged view illustrating a part (FIG. 1: inside the dashed line frame) of the guide wire on a distal end side.

FIG. 3 is an explanatory view illustrating a transverse sectional configuration taken along line A-A in FIG. 2.

FIGS. 4A, 4B, 4C, and 4D are each an explanatory diagram illustrating a method for manufacturing the guide wire as the medical device.

FIGS. 5A, 5B, and 5C are each an explanatory diagram illustrating the method for manufacturing the guide wire as the medical device.

FIGS. 6A, 6B, and 6C are each an explanatory diagram illustrating the method for manufacturing the guide wire as the medical device.

FIG. 7 is an enlarged view illustrating a part of a guide wire according to the second embodiment on a distal end side.

FIG. 8 is an enlarged view illustrating a part of a guide wire according to the third embodiment on a distal end side.

FIG. 9 is an explanatory view illustrating a transverse sectional configuration of a guide wire according to the fourth embodiment, taken along line A-A (FIG. 1).

### DETAILED DESCRIPTION

### First Embodiment

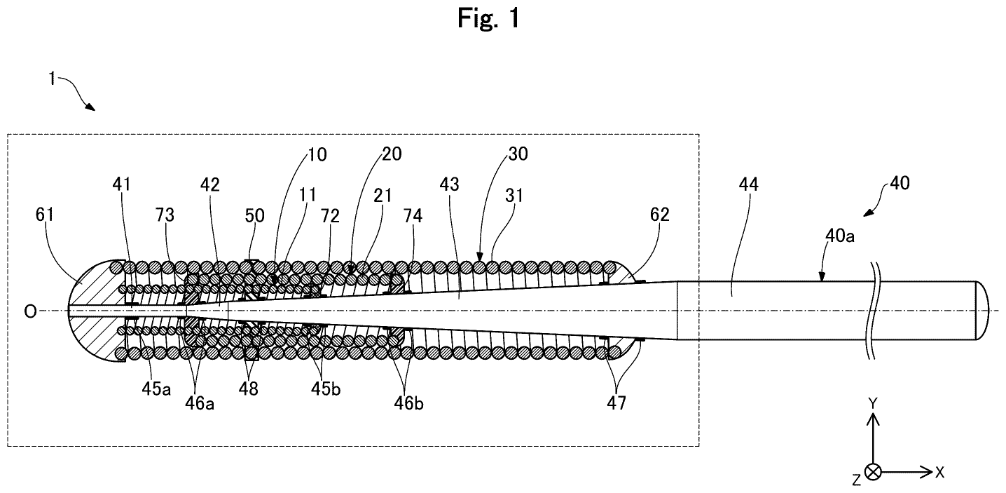

FIG. 1 is an explanatory view illustrating a configuration of a guide wire 1 as a medical device. FIG. 1 illustrates a longitudinal sectional configuration of the guide wire 1. The guide wire 1 is a medical device that is inserted into a blood vessel or the like. The guide wire 1 includes a first inner coil 10, a second inner coil 20, an outer coil 30, a core shaft 40, an intermediate fixing member 50, a distal end fixing member 61, a proximal end fixing member 62, a first proximal end fixing member 72, a second distal end fixing member 73, and a second proximal end fixing member 74. Although explanation will be made with reference to a blood vessel in the following examples, the guide wire 1 can be inserted into living body lumens such as lymph gland system, bile tract system, urinary system, respiratory tract system, digestive organ system, secreting gland system, and reproductive organs besides the vascular system, for use.

In FIG. 1, an axis passing through the center of the guide wire 1 is represented by an axis line O (dashed-dotted line). In the example of FIG. 1, the axis line O coincides with each axis passing through each center of the first inner coil 10, the second inner coil 20, the outer coil 30, and the core shaft 40. However, the axis line O may be deviated from each central axis of the components described above. FIG. 1 illustrates X, Y, and Z axes orthogonal to each other. The X axis corresponds to the longitudinal direction of the guide wire 1, the Y axis corresponds to the height direction of the guide wire 1, and the Z axis corresponds to the width direction of the guide wire 1. The left side (−X axis direction) in FIG. 1 is referred to as “distal end side” of the guide wire 1 and each component, and the right side (+X axis direction) in FIG. 1 is referred to as “proximal end side” of the guide wire 1 and each component. Among both ends of the guide wire 1 and each component in the longitudinal direction (X axis direction), one end located on the distal end side is referred to as “distal end”, and the other end located on the proximal end side is referred to as “proximal end”. The distal end and the vicinity thereof are referred to as “distal end portion,” and the proximal end and the vicinity thereof are referred to as “proximal end portion”. The distal end side is inserted into a living body, and the proximal end side is operated by an operator such as a surgeon. These configurations are common with the figures following FIG. 1.

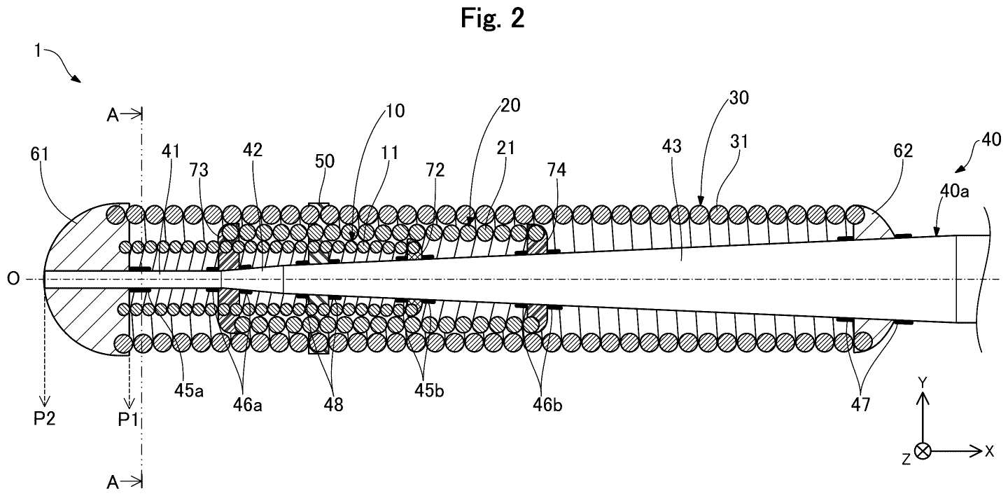

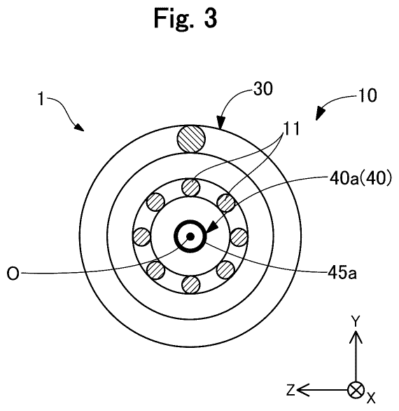

FIG. 2 is an enlarged view illustrating a part (FIG. 1: inside the dashed line frame) of the guide wire 1 on the distal end side. FIG. 3 is an explanatory view illustrating a transverse sectional configuration taken along line A-A in FIG. 2.

As illustrated in FIG. 1, the core shaft 40 has an elongated outer shape extending along the axis line O. The core shaft 40 includes a small diameter portion 41, a first tapered portion 42, a second tapered portion 43, a large diameter portion 44, a first distal end coating film 45a, first proximal end coating films 45b, second distal end coating films 46a, second proximal end coating films 46b, third coating films 47, and fourth coating films 48 in this order from the distal end toward the proximal end. Portions constituting the main body of the core shaft 40, specifically, the small diameter portion 41, the first tapered portion 42, the second tapered portion 43, and the large diameter portion 44 are collectively referred to as “core shaft main body 40a”. The core shaft 40 corresponds to “first metal member” or “second metal member”. The core shaft main body 40a corresponds to “first metal member main body”. The coating films provided on the core shaft main body 40a, specifically the first distal end coating film 45a, the first proximal end coating films 45b, the second distal end coating films 46a, the second proximal end coating films 46b, the third coating films 47, and the fourth coating films 48 are collectively referred to as “coating films”.

The small diameter portion 41 is disposed on the frontmost end side of the core shaft 40. The small diameter portion 41 has an elongated shape extending coaxially with the axis line O of the guide wire 1 (FIG. 1, FIG. 2), and is a columnar portion with a circular transverse section as illustrated in FIG. 3. The distal end of the small diameter portion 41 is fixed to the first inner coil 10 and the outer coil 30 by a distal end fixing member 61. A first tapered portion 42 is connected to the proximal end of the small diameter portion 41. The outer diameter, the axis line O direction-length, and the transverse sectional shape of the small diameter portion 41 can be arbitrarily determined. For example, the small diameter portion 41 may have a flat plate outer shape with a Y axis direction length smaller than a Z axis direction length in the transverse section illustrated in FIG. 3. In this case, the small diameter portion 41 can be referred to as “flat prate portion”. The small diameter portion 41 is not necessarily coaxial with the first tapered portion 42 and the large diameter portion 44. In this case, one side surface of the small diameter portion 41 on the proximal end side may be joined with one side surface of the first tapered portion 42 on the distal end side.

The first tapered portion 42 is disposed between the small diameter portion 41 and the second tapered portion 43. The first tapered portion 42 has an almost circular truncated cone shape with an outer diameter gradually reducing from the proximal end toward the distal end. As illustrated in FIG. 1, the small diameter portion 41 is connected to the distal end of the first tapered portion 42, and the second tapered portion 43 is connected to the proximal end of the first tapered portion 42. The outer diameter, the axis line O direction-length, and the transverse sectional shape of the first tapered portion 42 can be arbitrarily determined.

The second tapered portion 43 is disposed between the first tapered portion 42 and the large diameter portion 44. The second tapered portion 43 has an almost circular truncated cone shape with an outer diameter gradually reducing from the proximal end toward the distal end. As illustrated in FIG. 1, the first tapered portion 42 is connected to the distal end of the second tapered portion 43, and the large diameter portion 44 is connected to the proximal end of the second tapered portion 43. The outer diameter, the axis line O direction-length, and the transverse sectional shape of the second tapered portion 43 can be arbitrarily determined. In the example of the figure, the second tapered portion 43 has an axis line O direction-length larger than of the first tapered portion 42, and a taper angle smaller than of the first tapered portion 42.

The large diameter portion 44 is disposed on the most proximal end side of the core shaft 40. The large diameter portion 44 has an almost columnar shape with a substantially constant outer diameter from the proximal end to the distal end. The large diameter portion 44 has an outer diameter equal to the largest diameter of the second tapered portion 43. Note that, in this embodiment, the term “equal” means being substantially the same and allows differences due to production errors and the like. As illustrated in FIG. 1, the second tapered portion 43 is connected to the distal end of the large diameter portion 44. The proximal end portion of the large diameter portion 44 is held and operated by an operator. The outer diameter, the axis line O direction-length, and the transverse sectional shape of the large diameter portion 44 can be arbitrarily determined.

The first inner coil 10 covers the distal end portion of the core shaft 40. Specifically, the first inner coil 10 is arranged so as to surround a part of the small diameter portion 41 on the proximal end side, the first tapered portion 42, and a part of the second tapered portion 43 on the distal end side. The distal end of the first inner coil 10 is fixed to the core shaft 40 and the outer coil 30 by the distal end fixing member 61. The proximal end of the first inner coil 10 is fixed to the core shaft 40 by the first proximal end fixing member 72. The average coil diameter of the first inner coil 10 (average diameter of the outer diameter and the inner diameter of the first inner coil 10) and the length of the first inner coil 10 can be arbitrarily determined. In the guide wire 1 illustrated in FIG. 1, the first inner coil 10 corresponds to the second metal member, “hollow member”, and “first hollow member”. When the core shaft 40 corresponds to the first metal member, the first inner coil 10 corresponds to “second metal member”. When the core shaft 40 corresponds to the second metal member, the first inner coil 10 corresponds to “first metal member”.

As illustrated in FIG. 3, the first inner coil 10 according to this embodiment is a multi-thread coil with eight wires 11 wound in a multi-thread manner and has an almost hollow cylindrical shape with a constant outer diameter. The first inner coil 10 is a multi-thread coil formed e.g. by a process in which eight wires 11 are tightly twisted so as to be in contact with each other on a core metal, then residual stress is removed using a known heat treatment method, and the core metal is drawn out. For the first inner coil 10, any form can be adopted, and for example, the number of the wires 11 constituting the first inner coil 10 is not limited to eight and may be arbitrarily determined. The first inner coil 10 is not limited to a multi-thread coil, but also may be a single thread coil formed by winding one wire in a single thread manner, a single thread twisted wire coil formed by winding, in a single thread manner, a twisted wire with a plurality of wires twisted, or a multi-thread twisted wire coil formed by winding, in a multi-thread manner, each of a plurality of twisted wires with a plurality of wires twisted.

The second inner coil 20 is disposed on the radially outer side with respect to the first inner coil 10 to surround a part of the first inner coil 10 on the proximal end side and a part of the core shaft 40 (in the example of the figure, a part of the second tapered portion 43). In the axis line O direction, the distal end of the second inner coil 20 is located between the distal end and proximal end of the first inner coil 10. The distal end of the second inner coil 20 is fixed to the first inner coil 10 and the core shaft 40 by the second distal end fixing member 73. The proximal end of the second inner coil 20 is located on the proximal end side with respect to the proximal end of the first inner coil 10 in the axis line O direction. The proximal end of the second inner coil 20 is fixed to the core shaft 40 by the second proximal end fixing member 74.

The second inner coil 20 is a single thread coil formed by winding one wire 21 in a single thread manner. However, the second inner coil 20 is not limited to a single thread coil and may be a multi-thread coil, a single thread twisted wire coil, or a multi-thread twisted wire coil. The average coil diameter of the second inner coil 20 (average diameter of the outer diameter and the inner diameter of the second inner coil 20) and the length of the second inner coil 20 can be arbitrarily determined.

The outer coil 30 is disposed on the radially outer side with respect to the second inner coil 20 to surround the first inner coil 10, the second inner coil 20, and a part of the core shaft 40 (in the example of the figure, a part of the second tapered portion 43). The distal end of the outer coil 30 is located at the same position as the distal end of the first inner coil 10 in the axis line O direction. The distal end of the outer coil 30 is fixed to the core shaft 40 and the first inner coil 10 by the distal end fixing member 61. The proximal end of the outer coil 30 is located on the proximal end side with respect to the proximal end of the second inner coil 20 in the axis line O direction. The proximal end of the outer coil 30 is fixed to the core shaft 40 by the proximal end fixing member 62. In the guide wire 1 illustrated in FIG. 1, the outer coil 30 corresponds to “second hollow member”.

The outer coil 30 is a single thread coil formed by winding one wire in a single thread manner. However, the outer coil 30 is not limited to a single thread coil and may be a multi-thread coil, a single thread twisted wire coil, or a multi-thread twisted wire coil. The average coil diameter of the outer coil 30 (average diameter of the outer diameter and the inner diameter of the outer coil 30) and the length of the outer coil 30 can be arbitrarily determined.

In the example of FIG. 1, the winding direction of the outer coil 30 is reverse to the winding directions of the first inner coil 10 and the second inner coil 20. However, the winding directions of the first inner coil 10, the second inner coil 20, and the outer coil 30 may be the same, or the winding direction of any one among them may differ from the winding directions of the other two. In the example of FIG. 1, the outer peripheral surface of the first inner coil 10 and the inner peripheral surface of the second inner coil 20 are in contact with each other, and the outer peripheral surface of the second inner coil 20 and the inner peripheral surface of the outer coil 30 are in contact with each other. However, the outer peripheral surface of the first inner coil 10 and the inner peripheral surface of the second inner coil 20 may be separated from each other, and the outer peripheral surface of the second inner coil 20 and the inner peripheral surface of the outer coil 30 may be separated from each other.

The intermediate fixing member 50 is disposed between the second distal end fixing member 73 and the first proximal end fixing member 72 in the axis line O direction to fix a part of the first inner coil 10, a part of the second inner coil 20, a part of the outer coil 30, and a part of the core shaft 40 (distal end portion of the second tapered portion 43). The distal end fixing member 61 is disposed on the distal ends of the first inner coil 10 and the outer coil 30 to fix the distal end of the first inner coil 10 (hollow member), the distal end of the outer coil 30, and the distal end of the core shaft 40. As illustrated in FIG. 2, the distal end fixing member 61 is joined to the core shaft 40 on the distal end side with respect to a distal end P1 of the first distal end coating film 45a disposed on the frontmost side among the coating films. In the guide wire 1 illustrated in FIG. 1, the distal end fixing member 61 corresponds to “fixing member”. The proximal end fixing member 62 is disposed on the proximal end of the outer coil 30 to fix the proximal end of the outer coil 30 and a part of the core shaft 40 (proximal end portion of the second tapered portion 43).

The first proximal end fixing member 72 is disposed on the proximal end of the first inner coil 10 to fix the proximal end of the first inner coil 10 and a part of the core shaft 40 (a part of the second tapered portion 43). The second distal end fixing member 73 is disposed on the distal end of the second inner coil 20 to fix the distal end of the second inner coil 20, a part of the first inner coil 10, a part of the core shaft 40 (the distal end portion of the first tapered portion 42). The second proximal end fixing member 74 is disposed on the proximal end of the second inner coil 20 to fix the proximal end of the second inner coil 20 and a part of the core shaft 40 (a part of the second tapered portion 43).

The coating films will be explained with reference to FIG. 2 and FIG. 3. In this embodiment, the core shaft 40 has coating films (specifically, first distal end coating film 45a, first proximal end coating films 45b, second distal end coating films 46a, second proximal end coating films 46b, third coating films 47, and fourth coating films 48) formed on the surface of the core shaft main body 40a.

The first distal end coating film 45a is a film that is located on the proximal end side of the distal end fixing member 61 to cover the surface of the core shaft main body 40a (small diameter portion 41). In the surface of the core shaft main body 40a, the first distal end coating film 45a is disposed on a portion adjacent to the distal end fixing member 61 on the proximal end side with respect to the distal end fixing member 61. As illustrated in FIG. 2, the distal end P1 of the first distal end coating film 45a is located on the proximal end side with respect to a distal end P2 of the core shaft 40. As illustrated in FIG. 3, the first distal end coating film 45a is provided throughout the circumferential direction of the core shaft main body 40a to cover the whole circumference of the core shaft main body 40a.

The first proximal end coating films 45b are films that are located on the both sides of the first proximal end fixing member 72 to cover the surface of the core shaft main body 40a (second tapered portion 43). In the surface of the core shaft main body 40a, the first proximal end coating films 45b are disposed on a portion adjacent to the first proximal end fixing member 72 on the distal end side with respect to the first proximal end fixing member 72, and on a portion adjacent to the first proximal end fixing member 72 on the proximal end side with respect to the first proximal end fixing member 72. Similarly to the first distal end coating film 45a, the first proximal end coating films 45b cover the whole circumference of the core shaft main body 40a.

The second distal end coating films 46a are films that are located on the both sides of the second distal end fixing member 73 to cover the surface of the core shaft main body 40a (small diameter portion 41 and first tapered portion 42). In the surface of the core shaft main body 40a, the second distal end coating films 46a are disposed on a portion adjacent to the second distal end fixing member 73 on the distal end side with respect to the second distal end fixing member 73, and on a portion adjacent to the second distal end fixing member 73 on the proximal end side with respect to the second distal end fixing member 73. The second proximal end coating films 46b are films that are located on the both sides of the second proximal end fixing member 74 to cover the surface of the core shaft main body 40a (second tapered portion 43). In the surface of the core shaft main body 40a, the second proximal end coating films 46b are disposed on a portion adjacent to the second proximal end fixing member 74 on the distal end side with respect to the second proximal end fixing member 74, and on a portion adjacent to the second proximal end fixing member 74 on the proximal end side with respect to the second proximal end fixing member 74. Similarly to the first distal end coating film 45a, the second distal end coating films 46a and the second proximal end coating films 46b cover the whole circumference of the core shaft main body 40a.

The third coating films 47 are films that are located on the both sides of the proximal end fixing member 62 to cover the surface of the core shaft main body 40a (second tapered portion 43). In the surface of the core shaft main body 40a, the third coating films 47 are disposed on a portion adjacent to the proximal end fixing member 62 on the distal end side with respect to the proximal end fixing member 62, and on a portion adjacent to the proximal end fixing member 62 on the proximal end side with respect to the proximal end fixing member 62. The fourth coating films 48 are films that are located on the both sides of the intermediate fixing member 50 to cover the surface of the core shaft main body 40a (second tapered portion 43). In the surface of the core shaft main body 40a, the fourth coating films 48 are disposed on a portion adjacent to the intermediate fixing member 50 on the distal end side with respect to the intermediate fixing member 50, and on a portion adjacent to the intermediate fixing member 50 on the proximal end side with respect to the intermediate fixing member 50. Similarly to the first distal end coating film 45a, the third coating films 47 and the fourth coating films 48 cover the whole circumference of the core shaft main body 40a.

In the core shaft 40, the core shaft main body 40a (small diameter portion 41, first tapered portion 42, second tapered portion 43, and large diameter portion 44) is made of a superelastic alloy. Examples of the superelastic alloy include NiTi alloys and iron-based superelastic alloys. The core shaft main body 40a according to this embodiment is made of an alloy containing the “first element” and the “third element”. Either the first element or the third element accounts for the largest part of the elements contained in the core shaft main body 40a. Either the first element or the third element accounts for the second largest part of the elements contained in the core shaft main body 40a. The first element is a base metal element. The first element is nickel. The third element is a base metal element. The third element is titanium. The core shaft 40 may be made of a material which is more easily plastically deformed than superelastic alloys. Examples of the material which is more easily plastically deformed than superelastic alloys include stainless steel alloys such as SUS304 and SUS316. Also in this case, the core shaft main body 40a contains iron as the “first element” which is a base metal element and chromium or nickel as the “third element” which is a base metal element.

The coating films (first distal end coating film 45a, first proximal end coating films 45b, second distal end coating films 46a, second proximal end coating films 46b, third coating films 47, and fourth coating films 48) are formed in a manufacturing process using a flux containing at least one of tin (II) chloride (SnCl₂) and zinc chloride (ZnCl₂). The coating film is also formed on the surface of the first inner coil 10. The first inner coil 10 includes a first inner coil main body and the coating film formed on the surface of the first inner coil body. The longitudinal position of the coating film formed on the surface of the first inner coil 10 substantially coincides with the longitudinal position of each coating film formed on the core shaft 40. The coating films according to this embodiment contain at least one of tin (Sn) and zinc (Zn), which are metal elements different from all of the first, second, and third elements. For example, when the coating films are formed using a flux containing tin (II) chloride, the coating films include an oxide film containing tin (II) oxide (SnO). That means, the coating films according to this embodiment are oxide films. The formation of the coating films is described later.

The wire 11 constituting the first inner coil 10, the wire 21 constituting the second inner coil 20, and a wire 31 constituting the outer coil 30 can be made of any material. In this embodiment, the wire 11, the wire 21, and the wire 31 are made of SUS316. The wire 11, the wire 21, and the wire 31 may be made of e.g. a stainless steel alloy such as SUS304, a superelastic alloy such as NiTi alloy, a piano wire, a nickel-chromium alloy, a radiolucent alloy such as a cobalt alloy, gold, platinum, tungsten, and a radiopaque alloy such as an alloy containing these elements (e.g. a platinum-nickel alloy). The wire 11, the wire 21, and the wire 31 may be made of a same material or may be made of different materials. The size relation in the diameters between the wire 11, the wire 21, and the wire 31 may also be arbitrarily determined. The first inner coil 10 is made of an alloy containing the first element and the third element. Either the first element or the third element accounts for the largest part of the elements contained in the first inner coil 10. Either the first element or the third element accounts for the second largest part of the elements contained in the first inner coil 10. The first element is a base metal element. The first element is iron. The third element is a base metal element. The third element is chromium.

The intermediate fixing member 50, the distal end fixing member 61, the proximal end fixing member 62, the first proximal end fixing member 72, the second distal end fixing member 73, and the second proximal end fixing member 74 are all made of a gold-tin braze (SnAu) or a silver-tin braze (SnAg). For example, the silver-tin braze contains 96.5% of Sn and 3.5% of Ag. For example, the gold-tin braze contains 20% of Sn and 80% of Au. That means, in this embodiment, the intermediate fixing member 50, the distal end fixing member 61, the proximal end fixing member 62, the first proximal end fixing member 72, the second distal end fixing member 73, and the second proximal end fixing member 74 are different from both the first element (nickel) and the third element (titanium), and contain gold or silver as a “second element” which is a D-block element. The “D-block element” means a Group 3 to 12 element in the periodic table (excluding lanthanide elements and actinide elements). In other words, the D-block element means a transition element excluding lanthanide elements and actinide elements, and means a zinc group element. The “second element” in this embodiment is a transition element and a noble metal element. The “noble metal element” means eight elements of gold (Au), silver (Ag), platinum (Pt), palladium (Pd), rhodium (Rh), iridium (Ir), ruthenium (Ru), and osmium (Os). All of the intermediate fixing member 50, the distal end fixing member 61, the proximal end fixing member 62, the first proximal end fixing member 72, the second distal end fixing member 73, and the second proximal end fixing member 74 contain a fourth element. In this embodiment, the fourth element is tin.

As described above, in the guide wire 1 (medical device) according to the first embodiment, the core shaft main body 40a contains the first element, the distal end fixing member 61 (fixing member) contains the second element that is different from the first element and is a D-block element, and the coating films (specifically, first distal end coating film 45a, first proximal end coating films 45b, second distal end coating films 46a, second proximal end coating films 46b, third coating films 47, and fourth coating films 48) are formed on at least a part of the surface of the core shaft main body 40a. Thus, galvanic corrosion of the core shaft main body 40a can be suppressed by the coating films.

In the guide wire 1 according to the first embodiment, since the second element contained in the distal end fixing member 61 is a noble metal element, corrosion of the distal end fixing member 61 can be suppressed. Furthermore, when the second element is a metal element or silver, a melting point of the braze material can be lowered.

As described above, when the core shaft main body 40a contains base metal elements as the first and third elements and the distal end fixing member 61 (fixing member) contains a noble metal element as the second element, the galvanic corrosion is likely to progress due to the large difference in the ionization tendency between the base metal element and the noble metal element. In the guide wire 1 (medical device) according to the first embodiment, the galvanic corrosion of the core shaft main body 40a that is more prone to corrosion can be suppressed by the coating films.

In the guide wire 1 according to the first embodiment, since the coating films are oxide films, the effect of the coating films for suppressing the galvanic corrosion can be further improved.

In the guide wire 1 (medical device) according to the first embodiment, the distal end of the coating film, specifically the distal end P1 of the first distal end coating film 45a is located on the proximal end side with respect to the distal end P2 of the core shaft 40 (FIG. 2). Thus, the joint between the core shaft 40 and the distal end fixing member 61 (fixing member) can be prevented from being hindered by the first distal end coating film 45a (coating film). In other words, the joint strength between the core shaft 40 and the distal end fixing member 61 can be improved.

The guide wire 1 (medical device) according to the first embodiment further includes an outer coil 30 (second hollow member) that covers the first inner coil 10 (first hollow member). The distal end fixing member 61 (fixing member) is joined to the core shaft 40 at least on the distal end side with respect to the distal end P1 of the first distal end coating film 45a (coating film) (FIG. 2). Thus, the joint between the core shaft 40 and the distal end fixing member 61 can be prevented from being hindered by the first distal end coating film 45a. In other words, the joint strength between the core shaft 40 and the distal end fixing member 61 can be improved.

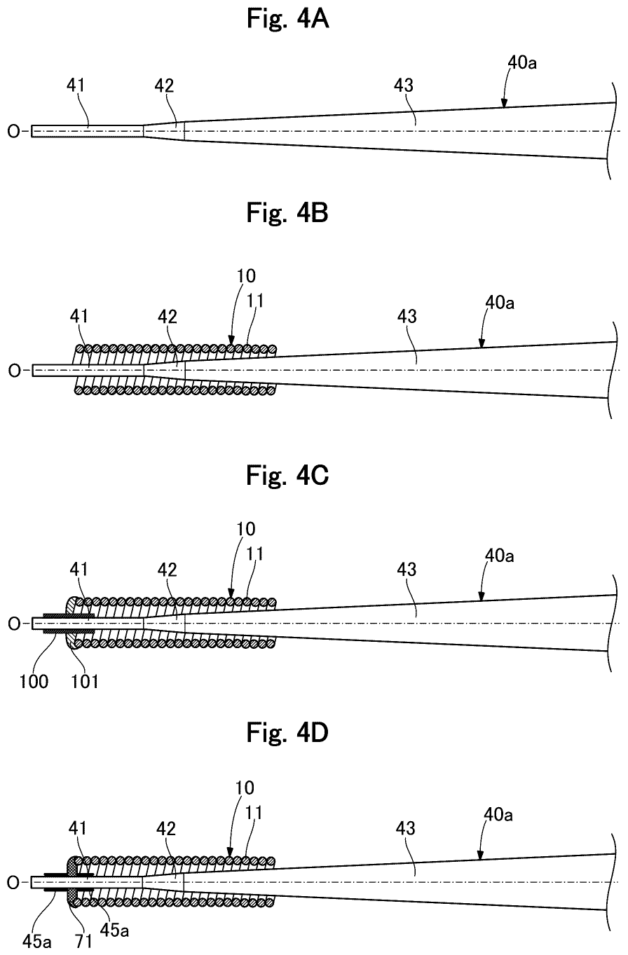

FIGS. 4A to 4D, FIGS. 5A to 5C, and FIGS. 6A to 6C are explanatory diagrams illustrating a method for manufacturing the guide wire 1 as a medical device. FIG. 4A is a diagram illustrating the core shaft main body 40a. FIG. 4B is a diagram illustrating an arrangement of the first inner coil 10. FIG. 4C is a diagram illustrating an arrangement of a braze material 101. FIG. 4D is a diagram illustrating a state that the first distal end fixing member 71 is formed.

First, as illustrated in FIG. 4A, a core shaft main body 40a containing the small diameter portion 41, the first tapered portion 42, the second tapered portion 43, and the large diameter portion 44 is prepared. The core shaft main body 40a is made of a NiTi alloy. The core shaft main body 40a may be made of a stainless steel alloy.

Next, as illustrated in FIG. 4B, the first inner coil 10 is disposed on the core shaft main body 40a so as to cover a part of the core shaft main body 40a on the distal end side (specifically, a part of the small diameter portion 41 on the proximal end side, the first tapered portion 42, and a part of the second tapered portion 43 on the distal end side). In the method for manufacturing the guide wire 1, the first inner coil 10 corresponds to the “first metal member”, the “hollow member”, and the “first hollow member”.

Subsequently, as illustrated in FIG. 4C, a flux 100 is applied, at a position corresponding to the distal end of the first inner coil 10, onto the surface of the core shaft main body 40a. The flux 100 contains tin (II) chloride (SnCl₂). The flux 100 may contain zinc chloride (ZnCl₂). After applying the flux 100, the braze material 101 is placed at a position corresponding to the distal end of the first inner coil 10. The braze material 101 is a silver-tin braze (SnAg). A gold-tin braze (SnAu) may be used as the braze material 101. After placing the braze material 101, a portion where the braze material 101 is placed is heated using a handgun or the like.

Consequently, as illustrated in FIG. 4D, the first distal end fixing member 71 is formed on the portion where the braze material 101 is placed, and the first distal end coating films 45a are formed on the both sides of the first distal end fixing member 71 in the surface of the core shaft main body 40a. Specifically, in the surface of the core shaft main body 40a, the first distal end coating films 45a are formed on a portion adjacent to the first distal end fixing member 71 on the distal end side with respect to the first distal end fixing member 71, and on a portion adjacent to the first distal end fixing member 71 on the proximal end side with respect to the first distal end fixing member 71. In the method for manufacturing the guide wire 1, FIGS. 4C and 4D correspond to the “forming step”, and the first distal end fixing member 71 corresponds to the “fixing member” and the “first fixing member”. Hereinafter, a series of steps in which the flux 100 is applied onto the surface of the core shaft main body 40a, on which the braze material 101 is placed and heated, is also referred to as “brazing step”.

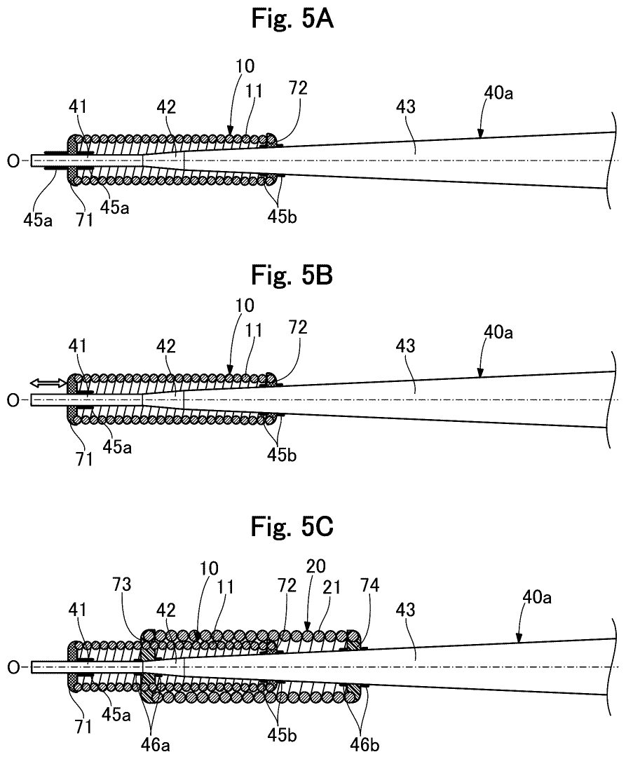

FIG. 5A is a diagram illustrating a state that the first proximal end fixing member 72 is formed. FIG. 5B is a diagram illustrating a state that the first distal end coating film 45a on the distal end side is removed. FIG. 5C is a diagram illustrating a state that the second inner coil 20 is placed and fixed.

As illustrated in FIG. 5A, brazing is performed at a position corresponding to the proximal end of the first inner coil 10 to form the first proximal end fixing member 72 and the first proximal end coating films 45b.

Subsequently, as illustrated in FIG. 5B, the first distal end coating film 45a formed on the distal end side with respect to the first distal end fixing member 71 (fixing member), i.e. the first distal end coating film 45a in the range indicated by the white blank arrow in FIG. 5B is removed. In the example of the figure, the whole first distal end coating film 45a formed on the distal end side with respect to the first distal end fixing member 71 is removed, but herein, it is sufficient to remove at least a part of the first distal end coating film 45a formed on the distal end side with respect to the first distal end fixing member 71. In the method for manufacturing the guide wire 1, FIG. 5B corresponds to the “removing step”.

Subsequently, as illustrated in FIG. 5C, the second inner coil 20 is arranged so as to cover the first inner coil 10. Then, brazing is performed at a position corresponding to the distal end of the second inner coil 20 to form the second distal end fixing member 73 and the second distal end coating films 46a. Also, brazing is performed at a position corresponding to the proximal end of the second inner coil 20 to form the second proximal end fixing member 74 and the second proximal end coating films 46b.

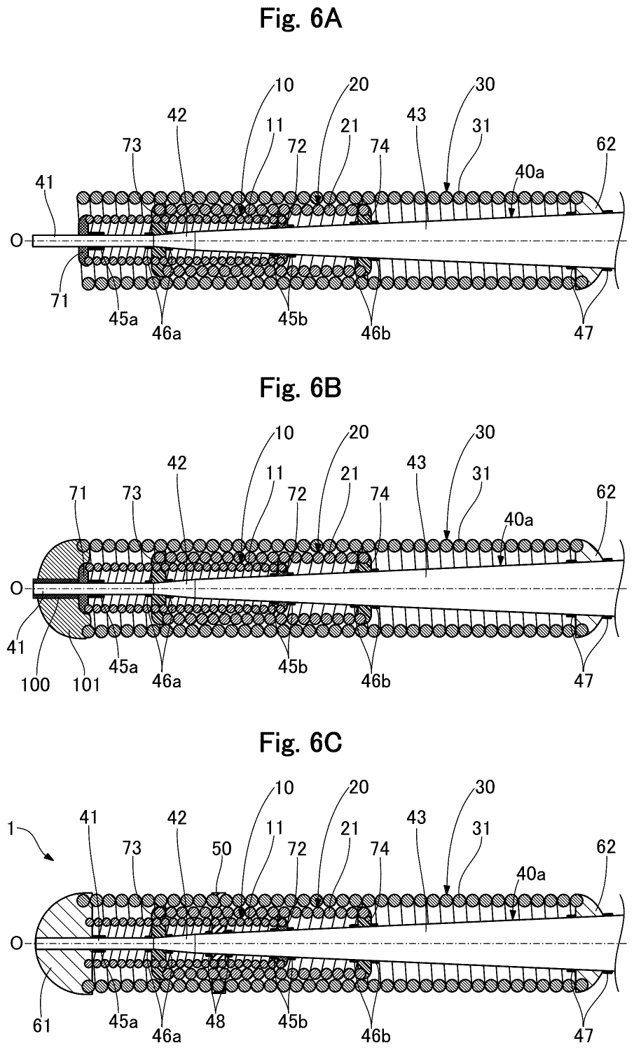

FIG. 6A is a diagram illustrating a state that the outer coil 30 is placed and fixed. FIG. 6B is a diagram illustrating an arrangement of the braze material 101. FIG. 6C is a diagram illustrating a state that the distal end fixing member 61 is formed.

As illustrated in FIG. 6A, the outer coil 30 is arranged so as to cover the first inner coil 10 and the second inner coil 20. In the example of the figure, the outer coil 30 is arranged such that the distal end position of the outer coil 30 and the distal end position of the first distal end fixing member 71 coincide with each other. Then, brazing is performed at a position corresponding to the proximal end of the outer coil 30 to form the proximal end fixing member 62 and the third coating films 47. In the method for manufacturing the guide wire 1, the outer coil 30 corresponds to the “second hollow member”.

Subsequently, as illustrated in FIG. 6B, the flux 100 is applied onto an area of the core shaft main body 40a surface, from which the first distal end coating film 45a has been removed in the removing step of FIG. 5B. After applying the flux 100, the braze material 101 is placed at a position corresponding to the distal end of the outer coil 30. As illustrated in the figure, the braze material 101 is arranged so as to encompass at least a part of the first distal end fixing member 71 (in the example of the figure, the whole part excluding the proximal end surface). After placing the braze material 101, the portion where the braze material 101 has been placed is heated using a handgun or the like.

Consequently, as illustrated in FIG. 6C, when braze material 101 and the first distal end fixing member 71 are molten and then hardened on the portion where the braze material 101 is placed, the distal end fixing member 61 is formed thereon. In the method for manufacturing the guide wire 1, FIGS. 6B and 6C correspond to the “second forming step”, and the distal end fixing member 61 corresponds to the “second fixing member”. If there is a protruding portion of the core shaft main body 40a (small diameter portion 41) on the distal end side with respect to the distal end fixing member 61 after the second forming step, the protruding portion of the core shaft main body 40a is removed by cutting it off as illustrated in FIG. 6C. Then, brazing is performed at a position corresponding to the intermediate position between the second distal end fixing member 73 and the first proximal end fixing member 72 to form the intermediate fixing member 50 and the fourth coating films 48.

The mechanism for forming the coating films (specifically, first distal end coating film 45a, first proximal end coating films 45b, second distal end coating films 46a, second proximal end coating films 46b, third coating films 47, and fourth coating films 48) will be explained. In the manufacturing method described above, when the first distal end fixing member 71, the first proximal end fixing member 72, the second distal end fixing member 73, the second proximal end fixing member 74, the distal end fixing member 61, the proximal end fixing member 62, and the intermediate fixing member 50 are formed, the flux 100 is applied onto each location intended for forming each fixing member, on which the braze material 101 is placed and then heated.

At this time, chemical reactions presented in the following Formulas (1) to (3) occur. First, as presented in Formula (1), nickel (II) oxide (NiO) formed on the surface of the core shaft main body 40a is reduced by tin (II) chloride (SnCl₂) contained in the flux 100 to generate oxygen (O₂). At this time, as presented in Formula (2), tin (II) chloride (SnCl₂) contained in the flux 100 reacts with the generated oxygen (O₂) to produce tin oxide (SnO). Also, as presented in Formula (3), tin (Sn) contained in the braze material 101 reacts with the generated oxygen (O₂) to promote the production of tin oxide (SnO). For this reason, the coating films in this embodiment contain tin as the fourth element. As a result, oxide films containing tin (II) oxide (SnO) (specifically, first distal end coating film 45a, first proximal end coating films 45b, second distal end coating films 46a, second proximal end coating films 46b, third coating films 47, and fourth coating films 48) are formed. The same applies to the case using a gold-tin braze (SnAu) as the braze material 101. The coating film of the first inner coil 10 is also formed by the same chemical reactions as represented by Formulas (1) to (3). The O₂ supply represented by Formula (1) is based mainly on nickel oxide of the core shaft main body 40a also in the case of the first inner coil 10, and may be based on chromium oxide and iron oxide contained in the first inner coil 10. Also when using the flux 100 containing zinc chloride (ZnCl₂), an oxide film containing zinc oxide (ZnO) is formed by the same reaction.

2NiO→2Ni+O₂ (1)

2SnCl₂+O₂→2SnO+2Cl₂ (2)

2Sn+O₂→2SnO (3)

In this way, as illustrated in FIGS. 4C and 4D, the method for manufacturing the guide wire 1 (medical device) according to the first embodiment includes a forming step in which the flux 100 is applied onto the core shaft main body 40a containing the first element, and brazing is performed using the braze material 101 containing the second element that is different from the first element and is a D-block element to form the first distal end fixing member 71 (fixing member) and the first distal end coating film 45a (coating film) of the core shaft main body 40a. Thereby, the coating film can suppress the galvanic corrosion of, among the core shaft main body 40a and the first distal end fixing member 71 and distal end fixing member 61 containing different elements, one that is more prone to corrosion.

In the method for manufacturing the guide wire 1 (medical device) according to the first embodiment, the forming step illustrated in FIGS. 4C and 4D includes forming the first distal end coating film 45a (coating film) on a portion adjacent to the first distal end fixing member 71 on the distal end side with respect to the first distal end fixing member 71 (fixing member), and on a portion adjacent to the first distal end fixing member 71 on the proximal end side with respect to the first distal end fixing member 71, in the surface of the core shaft main body 40a. Thereby, the effect of the coating film for suppressing the galvanic corrosion can be further improved.

Furthermore, in the method for manufacturing the guide wire 1 (medical device) according to the first embodiment, the first distal end fixing member 71 (fixing member) is a member that fixes the distal end portion of the core shaft 40 (specifically, core shaft main body 40a) and the distal end portion of the first inner coil 10 (hollow member), and the method for manufacturing the guide wire 1 includes a removing step of removing at least a part of the first distal end coating film 45a (coating film) formed on the distal end side with respect to the first distal end fixing member 71, as illustrated in FIG. 5B. This makes it possible to remove the coating film (specifically, first distal end coating film 45a formed on the distal end side of the first distal end fixing member 71) that hinders the joint between the core shaft 40 (specifically, core shaft main body 40a) and the distal end fixing member 61 (second fixing member).

Furthermore, the method for manufacturing the guide wire 1 (medical device) according to the first embodiment include a second forming step in which the flux 100 is applied onto a portion of the core shaft main body 40a from which the first distal end coating film 45a (coating film) has been removed in the removing step, on which brazing is performed using the braze material 101 containing the second element, to form the distal end fixing member 61 (second fixing member), as illustrated in FIGS. 6B and 6C. Thereby, the joint between the core shaft 40 (specifically, core shaft main body 40a) and the distal end fixing member 61 can be prevented from being hindered by the coating film (specifically, first distal end coating film 45a formed on the distal end side with respect to the first distal end fixing member 71). In other words, the joint strength between the core shaft 40 and the distal end fixing member 61 can be improved.

### Second Embodiment

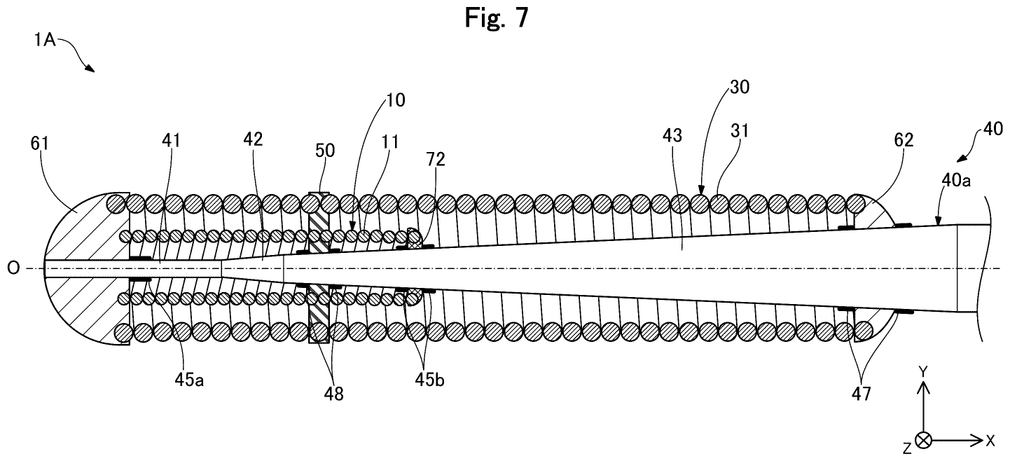

FIG. 7 is an enlarged view illustrating a part of a guide wire 1A according to the second embodiment on the distal end side. The guide wire 1A according to the second embodiment does not include the second inner coil 20 in the configuration described in the first embodiment. The guide wire 1A does not include the second distal end fixing member 73 and second proximal end fixing member 74 for fixing the second inner coil 20, the second distal end coating films 46a formed in association with the formation of the second distal end fixing member 73, and the second proximal end coating films 46b formed in association with the formation of the second proximal end fixing member 74.

As described above, the configuration of the guide wire 1A can be variously modified, and the guide wire 1A may be configured without the second inner coil 20. In the example of FIG. 7, the first inner coil 10 and the outer coil 30 are disposed apart from each other in the circumferential direction of the guide wire 1A, but the outer peripheral surface of the first inner coil 10 and the inner peripheral surface of the outer coil 30 may be in contact with each other. In the configuration of FIG. 7, the outer coil 30, the proximal end fixing member 62 for fixing the outer coil 30, and the third coating films 47 may be omitted. In the configuration of FIG. 7, besides the outer coil 30, the proximal end fixing member 62, and the third coating films 47, the intermediate fixing member 50 and the fourth coating films 48 may be omitted. The same effect as in the first embodiment described above can be provided also in the guide wire 1A according to the second embodiment.

### Third Embodiment

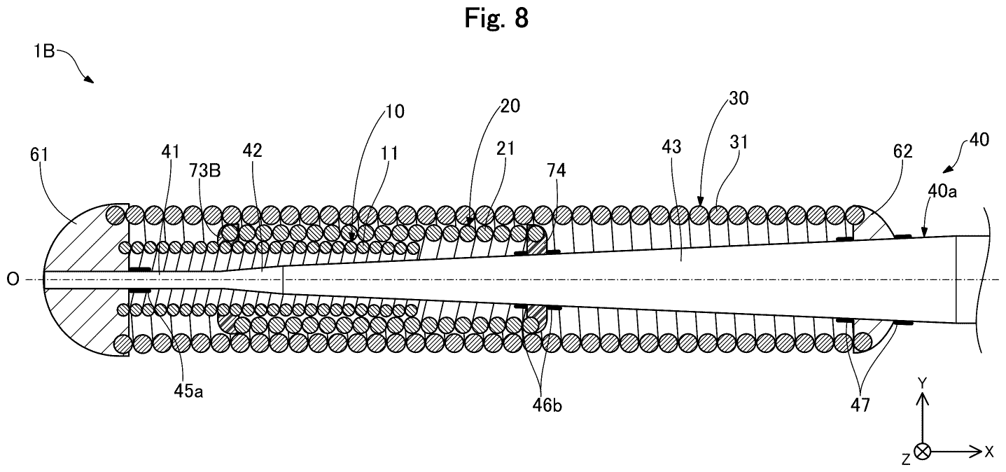

FIG. 8 is an enlarged view illustrating a part of a guide wire 1B according to the third embodiment on the distal end side. The guide wire 1B according to the third embodiment does not include the first proximal end fixing member 72 and the intermediate fixing member 50, but includes a second distal end fixing member 73B instead of the second distal end fixing member 73. The guide wire 1B does not include the first proximal end coating films 45b formed in association with the formation of the first proximal end fixing member 72 and the fourth coating films 48 formed in association with the formation of the intermediate fixing member 50, and furthermore does not include the second distal end coating films 46a.

In the guide wire 1B, the proximal end of the first inner coil 10 is not fixed to the core shaft 40. The second distal end fixing member 73B fixes the distal end of the second inner coil 20 and a part of the first inner coil 10 but is not joined to the core shaft 40. During arrangement and fixing of the second inner coil 20 as explained in FIG. 5C of the first embodiment, if the flux 100 and the braze material 101 do not reach the core shaft main body 40a but remain on the surface of the first inner coil 10, the second distal end fixing member 73B as illustrated in FIG. 8 is formed.

As described above, the configuration of the guide wire 1B can be variously modified, and may be configured without the first proximal end fixing member 72 and the intermediate fixing member 50. The second distal end fixing member 73B need not be joined to the core shaft 40. FIG. 8 is merely an example, and the second distal end fixing member 73B or the second proximal end fixing member 74 may be omitted instead of the first proximal end fixing member 72 or together with the first proximal end fixing member 72. Furthermore, the intermediate fixing member 50 may be or may not be omitted. The same effect as in the first embodiment described above can be provided also in the guide wire 1B according to the third embodiment.

### Fourth Embodiment

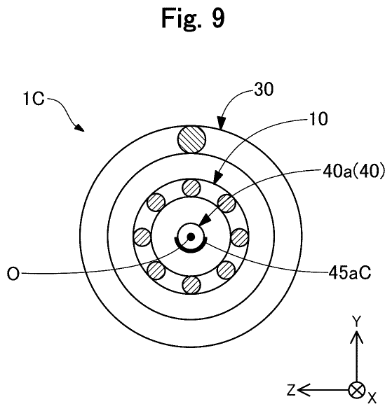

FIG. 9 is an explanatory view illustrating a transverse sectional configuration of a guide wire 1C according to the fourth embodiment, taken along line A-A (FIG. 1). The guide wire 1C according to the fourth embodiment includes a first distal end coating film 45aC instead of the first distal end coating film 45a. As illustrated in FIG. 9, the first distal end coating film 45aC is provided on a part (in the example of the figure, about a half part) of the core shaft main body 40a in the circumferential direction to cover a part of the surface of the core shaft main body 40a.

As described above, the configuration of the coating film can be variously modified, and the coating film only needs to cover at least a part of the core shaft main body 40a, and does not necessarily cover the entire circumference of the core shaft main body 40a. In the example of FIG. 9, the configuration of the first distal end coating film 45a was described as an example, but this configuration also applies to the coating films other than the first distal end coating film 45a (specifically, first proximal end coating films 45b, second distal end coating films 46a, second proximal end coating films 46b, third coating films 47, and fourth coating films 48). The same effect as in the first embodiment described above can be provided also in the guide wire 1C according to the fourth embodiment.

### MODIFICATIONS OF EMBODIMENTS

The disclosed embodiments are not limited to the above-described embodiments and may be implemented in various modes without departing from the gist thereof, and for example, the following modifications are also possible.

### [Modification 1]

In the first to fourth embodiments described above, the configurations of guide wires 1 and 1A to 1C have been described as examples. However, the configurations of the guide wires 1 and 1A to 1C can be variously modified. For example, the core shaft main body of the guide wire 1 may arbitrarily include a small diameter portion, a large diameter portion, a flat portion, a tapered portion, or the like depending on performances required for the guide wire 1, and need not include at least some of the small diameter portion 41, the first tapered portion 42, the second tapered portion 43, and the like described above.

### [Modification 2]

In the first to fourth embodiments described above, the configurations of the first inner coil 10, the second inner coil 20, and the outer coil 30 have been described as examples. However, these configurations can be variously modified. For example, the outer coil 30 may be configured so as not to be a part of the core shaft 40 on the distal end side but to be cover the entire core shaft 40. In other words, the proximal end of the outer coil 30 may extend to the proximal end of the core shaft 40. For example, one or more of the first inner coil 10, the second inner coil 20, and the outer coil 30 may be formed from an almost hollow cylindrical tube instead of the coil with a wire spirally wound. In this case, the tube may have a tubular shape with a slit that penetrates the inside and outside of the tube or without any slit. A reinforcing member with wires woven in a mesh form or a coil-shaped reinforcing member may be embedded in the tube.

### [Modification 3]

In the first to fourth embodiments, the materials constituting the various members have been described as examples. However, the various members may be made of materials different from the above-described materials. For example, the core shaft main body may contain elements different from base metal elements. For example, at least some of the braze material 101, the first distal end fixing member 71, the first proximal end fixing member 72, the second distal end fixing member 73, the second proximal end fixing member 74, the distal end fixing member 61, the proximal end fixing member 62, and the intermediate fixing member 50 need not contain any transition element (e.g. may contain zinc) and need not contain noble metal elements (e.g. may contain copper). For example, at least some of the flux 100, the first distal end coating film 45a, the first proximal end coating films 45b, the second distal end coating films 46a, the second proximal end coating films 46b, the third coating films 47, and the fourth coating films 48 need not contain any metal element, e.g. tin or zinc. For example, at least some of the first distal end coating film 45a, the first proximal end coating films 45b, the second distal end coating films 46a, the second proximal end coating films 46b, the third coating films 47, and the fourth coating films 48 need not be an oxide film.

### [Modification 4]

The medical device may be a catheter, a basket, a snare, a stent, or the like. The coating film may be formed by plating, thermal spraying, vapor deposition, or the like. The fourth element contained in the coating film may be at least any of copper, bismuth, antimony, germanium, and aluminum. The coating film containing at least any of copper, bismuth, antimony, germanium, and aluminum may be formed from a flux or by the above-described plating, thermal spraying, vapor deposition, or the like.

### [Modification 5]

The configurations of the guide wires according to the first to fourth embodiments and the configurations of the guide wires according to the above-described modifications 1 to 4 may be combined as appropriate. For example, the guide wires according to the second and third embodiments may be configured to include the coating film described in the fourth embodiment. For example, in the guide wire 1A according to the second embodiment, the intermediate fixing member 50 and the fourth coating films 48 may be omitted, or the first proximal end fixing member 72 and the first proximal end coating films 45b may be omitted.

The disclosed embodiments can include the following aspects.

(1) According to an aspect of the disclosed embodiments, a medical device is provided. This medical device includes: a first metal member including a first metal member main body including a first element, and a coating film formed on at least a part of a surface of the first metal member main body; a second metal member; and a fixing member that fixes the first metal member and the second metal member and including a second element that is different from the first element and is a D-block element.

According to this configuration, galvanic corrosion of, among the first metal member main body and the fixing member which include different elements, one that is more prone to corrosion can be suppressed by the coating film. That means, according to this configuration, the corrosion of the members constituting the medical device can be suppressed.
(2) The medical device according to the above aspect may be configured such that the second element is a transition element.
(3) The medical device according to the above aspects may be configured such that the second element is a noble metal element. According to this configuration, corrosion of the fixing member can be suppressed.
(4) The medical device according to the above aspects may be configured such that the first element is a base metal element.
(5) The medical device according to the above aspects may be configured such that the first metal member main body is an alloy containing a third element, and the coating film contains a fourth element that is different from all of the first, second, and third elements and belongs to Group 10 to Group 15 and Period 3 to Period 6.
(6) The medical device according to the above aspects may be configured such that the fourth element is at least one element of tin, zinc, copper, bismuth, antimony, germanium, and aluminum.
(7) The medical device according to the above aspects may be configured such that the fixing member contains the fourth element.
(8) The medical device according to the above aspects may be configured such that the coating film is an oxide film. According to this configuration, the effect of the coating film for suppressing galvanic corrosion can be further improved because the coating film is an oxide film.
(9) The medical device according to the above aspects may be configured such that the first metal member is a core shaft, the first metal member main body is a core shaft main body, and a distal end of the coating film is located on a proximal end side with respect to a distal end of the core shaft. According to this configuration, the joint between the core shaft and the fixing member can be prevented from being hindered by the coating film. In other words, the joint strength between the core shaft and the fixing member can be improved.
(10) The medical device according to the above aspects may be configured such that the second metal member is a first hollow member that covers the core shaft, the medical device further includes a second hollow member that covers the first hollow member, and the fixing member fixes the core shaft, the first hollow member, and the second hollow member and is joined to the core shaft at least on a distal end side with respect to a distal end of the coating film. According to this configuration, since the fixing member is joined to the core shaft at least on the distal end side with respect to the distal end of the coating film, the joint between the core shaft and the fixing member can be prevented from being hindered by the coating film. In other words, the joint strength between the core shaft and the fixing member can be improved.
(11) The medical device according to the above aspects may be configured such that the second metal member has a second metal member main body and a coating film formed on at least a part of a surface of the second metal member main body. According to this configuration, corrosion of the second metal member main body is suppressed by the coating film.
(12) According to an aspect of the disclosed embodiments, a method for manufacturing a medical device is provided, including a forming step in which a first metal member main body containing a first element and a second metal member that covers the first metal member main body are brazed using a flux and a braze material containing a second element that is different from the first element and is a D-block element, to form a fixing member that fixes the first metal member main body and the second metal member, and a coating film of the first metal member main body.
(13) The method for manufacturing the medical device according to the above aspect may be configured such that the first metal member main body is a core shaft main body, the core shaft main body and the coating film constitute a core shaft, the forming step includes forming the coating film on each of a portion adjacent to the fixing member on a distal end side with respect to the fixing member, and a portion adjacent to the fixing member on a proximal end side with respect to the fixing member, in a surface of the core shaft main body.
(14) The method for manufacturing the medical device according to the above aspects may be configured such that the second metal member is a hollow member that covers the core shaft, the fixing member fixes a distal end portion of the core shaft and a distal end portion of the hollow member, and the method for manufacturing the medical device further includes a removing step of removing at least a part of the coating film formed on the distal end side with respect to the fixing member. According to this configuration, it is possible to remove the coating film (specifically, the coating film formed on the distal end side with respect to the fixing member) that hinders the joint between the core shaft and a second fixing member.
(15) The method for manufacturing the medical device according to the above aspects may be configured such that, in a case that the hollow member is defined as a first hollow member and the fixing member is defined as a first fixing member, the method for manufacturing the medical device further includes a second forming step in which, after the removing step, brazing is performed using the braze material containing the second element to form a second fixing member on a portion of the core shaft main body from which the coating film has been removed in the removing step so that the core shaft main body is fixed with the first hollow member and second hollow member. According to this configuration, the joint between the core shaft and the second fixing member can be prevented from being hindered by the coating film (specifically, coating film formed on the distal end side with respect to the fixing member). In other words, the joint strength between the core shaft and the second fixing member can be improved.
(16) The method for manufacturing the medical device according to the above aspects may be configured such that the flux contains at least one element of tin, zinc, copper, bismuth, antimony, germanium, and aluminum.

The disclosed embodiments can be embodied in various forms, for example, in a form of a medical device, a guide wire, or a manufacturing method therefor.

The aspects of the disclosed embodiments have been described above on the basis of the embodiments and modifications, however, the embodiments of the aspects described above are intended to facilitate understanding of the aspects, and are not intended to limit the aspects. The aspects may be modified and improved without departing from the gist and the scope of claims and includes equivalents thereof. If the technical features of the disclosed embodiments are not described as essential in the present specification, the technical features may be deleted as appropriate.

### DESCRIPTION OF REFERENCE NUMERALS

1, 1A to 1C . . . Guide wire
10 . . . First inner coil
11, 21, 31 . . . Wire
20 . . . Second inner coil
30 . . . Outer coil
40 . . . Core shaft
40a . . . Core shaft main body
41 . . . Small diameter portion
42 . . . First tapered portion
43 . . . Second tapered portion
44 . . . Large diameter portion
45a, 45aC . . . First distal end coating film
45b . . . First proximal end coating film
46a . . . Second distal end coating film
46b . . . Second proximal end coating film
47 . . . Third coating film
48 . . . Fourth coating film
50 . . . Intermediate fixing member
61 . . . Distal end fixing member
62 . . . Proximal end fixing member
71 . . . First distal end fixing member
72 . . . First proximal end fixing member
73, 73B . . . Second distal end fixing member
74 . . . Second proximal end fixing member
100 . . . Flux
101 . . . Braze material

What is claimed is:

1. A medical device comprising:
a first metal member comprising a first metal member main body comprising a first element, and a coating film formed on at least a part of a surface of the first metal member main body;
a second metal member; and
a fixing member that fixes the first metal member and the second metal member and comprises a second element that is different from the first element and is a D-block element.

2. The medical device according to claim 1, wherein
the second element is a transition metal element.

3. The medical device according to claim 2, wherein
the second element is a noble metal element.

4. The medical device according to claim 1, wherein
the first element is a base metal element.

5. The medical device according to claim 1, wherein
the first metal member is an alloy comprising a third element, and
the coating film comprises a fourth element that is different from all of the first, second, and third elements, and the fourth element belongs to Group 10 to Group 15 and Period 3 to Period 6.

6. The medical device according to claim 5, wherein
the fourth element is at least one element of tin, zinc, copper, bismuth, antimony, germanium, and aluminum.

7. The medical device according to claim 5, wherein
the fixing member further comprises the fourth element.

8. The medical device according to claim 1, wherein
the coating film is an oxide film.

9. The medical device according to claim 1, wherein
the first metal member is a core shaft,
the first metal member main body is a core shaft main body, and
a distal end of the coating film is located on a proximal end side with respect to a distal end of the core shaft main body.

10. The medical device according to claim 9, wherein
the second metal member is a first hollow member that covers the core shaft,
the medical device further comprises a second hollow member that covers the first hollow member, and
the fixing member fixes the core shaft, the first hollow member, and the second hollow member and is joined to the core shaft at least on a distal end side with respect to a distal end of the coating film.

11. The medical device according to claim 10, wherein first hollow member and the second hollow member are each independently a single thread coil, a multi-thread coil, a single thread twisted wire coil, or a multi-thread twisted wire coil.

12. The medical device according to claim 1, wherein
the second metal member comprises a second metal member main body and a coating film formed on at least a part of a surface of the second metal member main body.

13. The medical device according to claim 1, wherein the fixing member includes a plurality of fixing members arranged along a length of the first metal member, and a plurality of coating films arranged adjacent to the fixing members along the length of the first metal member.

14. A method for manufacturing a medical device, comprising:
covering at least a part of a first metal member main body with a second metal member, the first metal member main body comprising a first element; and
brazing the first metal member main body and the second metal member by employing a flux and a braze material comprising a second element that is different from the first element and is a D-block element, to form a fixing member that fixes the first metal member main body and the second metal member, and a coating film on a surface of the first metal member main body.

15. The method according to claim 14, wherein
the first metal member main body is a core shaft main body,
the core shaft main body and the coating film constitute a core shaft, and
the coating film is formed on a surface of the core shaft main body adjacent to the fixing member on both a distal end side and a proximal end side with respect to the fixing member.

16. The method according to claim 15, wherein
the second metal member is a hollow member that covers the core shaft,
the fixing member fixes a distal end portion of the core shaft and a distal end portion of the hollow member, and
the method for manufacturing the medical device further comprises removing at least a part of the coating film formed on the distal end side with respect to the fixing member.

17. The method according to claim 16, further comprising:
covering at least a part of the hollow member, which is a first hollow member, with a second hollow member; and
after the removing of at least the part of the coating film, brazing the core shaft main body, the first hollow member, and the second hollow member by employing the braze material comprising the second element to form a second fixing member on a portion of the core shaft main body from which the coating film has been removed so that the core shaft main body is fixed with the first hollow member and second hollow member, wherein the fixing member is a first fixing member.

18. The method according to claim 14, wherein
the flux comprises at least one element of tin, zinc, copper, bismuth, antimony, germanium, and aluminum.

19. The method according to claim 12, wherein the flux includes a fourth element that is different from the first element and the second element, and the fourth element belongs to Group 10 to Group 15 and Period 3 to Period 6.

20. The method according to claim 19, wherein the braze material further includes the fourth element.
